(19) United States
(12) Patent Application Publication
(10) Pub. No.: US 2004/0215003 A1
(43) Pub. Date: Oct. 28, 2004

(54) Method for purifying and separating soy isoflavones

(76) Inventor: Thomas A. Dobbins, Howard, OH (US)

Correspondence Address: THOMPSON HINE L.L.P., 2000 COURTHOUSE PLAZA , N.E., 10 WEST SECOND STREET, DAYTON, OH, 45402, US

(21) Appl. No.: 10/706,296

(22) Filed: Nov. 12, 2003

Related U.S. Application Data
(60) Provisional application No. 60/425,541, filed on Nov. 12, 2002.

(51) Int. Cl. C07H 17/00

(52) U.S. Cl. 536/8

(57) ABSTRACT

A method for purifying soy isoflavones glycosides of genistin and daidzin from impurities present in a soy isoflavones concentrate. The method includes digesting a soy isoflavones concentrate with an acidic solution and separating insoluble solids from the acidic solution, wherein the solids are enriched in genistin and comprise glycosides of genistin and daidzin.

### CROSS REFERENCE TO RELATED APPLICATION

This application claims the benefit of U.S. Provisional Application Ser. No. 60/425,541 filed on Nov. 12, 2002.

### BACKGROUND OF THE INVENTION

Isoflavones are a unique class of phytoestrogens (plant hormones) that occur naturally in many plants, including soybeans (Glycine max). The three isoflavones found in soybeans are genistin, daidzin, and glycetin, typically in a ratio of genistin: daidzin: glycetin of 1.1:1.0:0.3.

It is widely anticipated that market demand for soy isoflavones will continue to grow. Scientists have demonstrated that isoflavones have the ability to inhibit cancer cell growth, and some researchers believe that isoflavones may contribute to soy's ability to lower blood-cholesterol levels.

Research shows that soy isoflavones have a wide range of health benefits that include moderating normal symptoms associated with menopause and promoting bone and heart health. It appears that about 100 milligrams of isoflavones (expressed in the glycoside form) are necessary to deliver most of these health benefits. This is about the average amount consumed daily by many Asians, who have a much lower incidence of heart disease, osteoporosis, and uncomfortable menopausal symptoms compared to Western societies.

Some women's health problems during and after middle age are related to a changing hormonal state. Consuming soy isoflavones can help moderate the natural hormonal changes associated with several menopausal and postmenopausal symptoms.

Soy isoflavones are potent anti-oxidants capable of reducing the amount of LDL-cholesterol (bad cholesterol) that undergoes modification in the body. Entry of the modified LDL-cholesterol into the walls of blood vessels contributes to the formation of plaques. These plaques cause the blood vessels to lose their ability to function normally. Research in both animals and humans shows that ingesting soy isoflavones can help maintain normal blood vessel function.

Soy isoflavones are actively studied for their effects on maintaining and improving bone health. Women can lose up to 15% of their total bone mass in the early years following the onset of menopause. This loss can be quite detrimental, particularly to women who enter menopause with weaker bones. Emerging research shows that isoflavones appear to play a role in both preventing bone loss and increasing bone density.

A body of research suggests that the pharmacological activities of the three soy isoflavones differ; it is widely believed that genistin is the most pharmacologically active soy isoflavone. Therefore, a method of separating genistin from daidzin and glycetin or of enriching the proportion of genistin to daidzin in soy isoflavone concentrates is desirable.

The isoflavones found in soybeans occur predominantly as glycosides (“glucones”) with only a minor aglycon (“aglucone”) content. The isoflavone glycosides genistin, daidzin and glycetin have a glucose molecule attached. The aglycons genistein, daidzein and glycitein do not include glucose. In addition, an appreciable percentage of the glucosides occur as malonates or acetates. The malonyl and acetyl moities of soy isoflavones glycosides are thermally labile, particularly at elevated pH (e.g., 9.5 to 11.0), and can be easily converted to the corresponding simple glycosides by digestion at moderate temperatures with sodium or potassium hydroxide or other alkalis.

Traditionally, the separation of genistin from daidzin and glycetin has required laborious ultrafiltration followed by preparative chromatography (as described in U.S. Pat. No. 5,679,806) or absorption onto and subsequent desorption from ion-exchange resins (as described in U.S. Pat. No. 6,020,471). These processes require handling large volumes of solutions owing to the modest solubility of soy isoflavones.

U.S. Pat. No. 5,702,752 teaches the use of various resins to adsorb the isoflavones from dilute aqueous solutions heated to various temperatures, taking advantage of the temperature-sensitive differential solubilities of isoflavones in order to effect their separation.

Commonly assigned provisional patent application Ser. No. 60/367,566 discloses that the calcium complex of genistin is markedly less soluble in various mixtures of polar organic solvents (e.g., acetone) and water than the corresponding calcium complexes of daidzin and glycetin, permitting the calcium complex of genistin to be separated and iteratively purified by filtration or centrifugation.

### SUMMARY OF THE INVENTION

In accordance with one embodiment of the invention a method of employing acidic solutions to separate the soy isoflavone glycosides genistin, daidzin, and glycetin from the impurities present in soy isoflavone concentrates is provided. The soy isoflavone concentrate is digested with the acidic solution and undissolved solids are removed by filtration or centrifugation. The acidic solution may include an inorganic mineral acid or an aliphatic organic acid. Co-solvents that are both freely miscible with the acid and chemically inert in its presence under the process conditions may also be present in the acidic solution. A particularly useful acidic solution includes hydrochloric acid and an alcoholic co-solvent having from 1 to 8 carbon atoms.

In accordance with another manifestation, the acidic solution comprises glacial acetic acid. The acidic solution may comprise a mixture of glacial acetic acid and a co-solvent that is both freely miscible with glacial acetic acid and chemically inert in its presence under the process conditions. Specific examples of useful co-solvents include alcohols containing from 1 to 12 carbon atoms, aliphatic hydrocarbons containing from 5 to 20 carbon atoms, aromatic hydrocarbons containing from 6 to 30 carbon atoms, ketones containing from 2 to 12 carbon atoms, esters containing from 3 to 30 carbon atoms and mixtures thereof.

Another manifestation of the present invention is a method of increasing the recovery of daidzin and glycetin by employing glacial acetic acid as the principal solvent in conjunction with co-solvents that reduce the polarity of glacial acetic acid, thereby reducing the solubilities of daidzin and glycetin. The co-solvents in accordance with certain aspects of the invention include alcohols containing from 1 to 12 carbon atoms, aliphatic hydrocarbons containing from 5 to 20 carbon atoms, aromatic hydrocarbons containing from 6 to 30 carbon atoms, ketones containing from 2 to 12 carbon atoms, esters containing from 3 to 30 carbon atoms and mixtures thereof.

In still another manifestation of the invention, a method of purifying soy isoflavone aglycons is provided. The method includes dissolving crude aglycon-containing mixtures in alkaline aqueous solutions at modest temperatures (typically below about 35° C.), removing insoluble impurities, lowering the pH of the solution, and collecting the purified soy isoflavone aglycons that precipitate. The pH of the alkaline aqueous solution in accordance with certain embodiments is from about 10 to about 14. The insoluble impurities may be removed from the alkaline aqueous solution by filtration or centrifugation in accordance with particular aspects of the invention. In a more particular aspect of the invention, the pH of the filtrate is adjusted to a value of 1 to 7. Recovery of the purified isoflavone aglycons from the acidified filtrate may be by filtration or centrifugation in accordance with certain aspects of the invention.

### DETAILED DESCRIPTION OF THE INVENTION

All documents cited are, in relevant part, incorporated herein by reference; the citation of any document is not to be construed as an admission that it is prior art with respect to the present invention.

As used herein, “separating” means the act or acts of separating a solid phase from a liquid phase by means including but not limited to filtration, centrifugation, settling, decanting, expressing, distillation, and combinations thereof, and wherein the means of separating may integrally include the further steps of (a) washing the solid phase with additional solution so as to remove entrained solutes and/or (b) drying the solid phase to evaporate residual liquid.

As used herein, “enriched in genistin” means that the ratio of genistin to daidzin is increased during processing of a given composition. In accordance with certain embodiments, the term refers to a composition is which the ratio is greater than about 3 to 1.

The bulk soy isoflavones concentrates currently available in the marketplace for use in formulating nutraceutical products and dietary supplements typically have a total nominal isoflavone content of 40% by weight; the isoflavones are present predominantly in their glycoside forms, although traces of aglycon may be present. Commercially available products include: “Solgen,” produced by Solbar Plant Extracts of Ashdod, Israel by a proprietary process; “Novasoy,” believed to be produced by Archer-Daniels-Midland according to the process described in U.S. Pat. No. 6,020,471; and “Prevastein,” believed to be produced by Central Soya according to the processes described in U.S. Pat. Nos. 6,228,993; 6,320,028; and 6,369,200.

Despite the differences in the methods of production and composition of these materials, they all respond similarly to the processes of the present invention, which flow from the discovery that the solubilities of the soy isoflavone glycosides genistin, daidzin, and glycetin and the impurities present in commercial soy isoflavone glycoside concentrates differ dramatically in acidic solutions. Acidic solutions useful herein include, but are not limited to, those acidic solutions containing an inorganic (“mineral”) acid or an aliphatic organic acid in combination with a co-solvent that is both freely miscible with the acid and chemically inert in its presence under the process conditions.

Specific examples of inorganic or mineral acids useful in accordance with the present invention include, but are not limited to, hydrochloric, hydrobromic, sulfuric, and phosphoric acids. In the case of mineral acids, suitable co-solvents include alcohols containing from 1 to 8 carbon atoms. In accordance with a specific embodiment of the present invention, acidic solutions comprising concentrated HCl and methanol at a volume ratio of from about 1:10 to about 1:5 are particularly useful.

Specific examples of aliphatic organic acids useful in accordance with the present invention include, but are not limited to, glacial acetic acid and formic acid. In the case of aliphatic organic acids, suitable co-solvents include alcohols containing 1 to 12 carbons, aliphatic hydrocarbons containing 1 to 20 carbons, aromatic hydrocarbons containing 6 to 30 hydrocarbons, ketones containing 2 to 12 carbons, and esters containing three to 30 carbons, and mixtures thereof. Specific examples of useful co-solvents include, but are not limited to, methanol, hexane, xylene, acetone and ethyl acetate. In accordance with a particular embodiment, acidic solvents containing from about 50% to 100% by volume aliphatic organic acid in admixture with various co-solvents can be used. Of course, those skilled in the art will realize that mixtures of other acids and co-solvents at other ratios may prove efficacious.

The ratio of soy isoflavones-containing material to acidic solution can vary and is not particularly limited. In accordance with one aspect of the invention, soy isoflavone glycosides are purified and separated by preparing a slurry of the soy isoflavones-containing material in a modest volume (typically 3:1 to 10:1 by weight) of the acidic solution and stirring to bring the impurities into solution. This digestion step can be performed over a wide range of temperatures depending upon the melting and freezing points of the acidic solvent employed, but it is generally efficacious at temperatures of from about 10 to 100° C., and can be conducted at ambient temperature (typically at from about 18 to 25° C.). In accordance with a particular embodiment of the invention, the digestion step is performed at ambient temperature. The undissolved solids are separated by any suitable method, typically by filtration or centrifugation, then dried to give a product of substantially higher purity.

Genistin is characteristically the least soluble soy isoflavone glycoside in the solvents of the present invention, so the purified soy isoflavone product will generally be enriched in genistin and depleted in daidzin and glycetin relative to the crude stating material. This property permits genistin or a genistin-enriched product to be selectively isolated.

However, in a further aspect of the present invention, it is possible to exert a high degree of control over the relative amounts of genistin, daidzin, and glycetin in the final product by employing glacial acetic acid as the principal solvent in conjunction with aliphatic hydrocarbon (e.g., hexane), aromatic hydrocarbon (e.g., xylene), ketone (e.g., acetone), or ester (e.g., ethyl acetate) co-solvents that serve to reduce the polarity of glacial acetic acid. Reducing the polarity of glacial acetic acid by the addition of co-solvents such as hexane, acetone, or ethyl acetate increases the amount of daidzin and glycetin recovered from the starting, material without adversely affecting the amount of genistin that is recovered. In a particular embodiment of the invention, a solvent mixture comprising about 80% glacial acetic acid and about 20% acetone is particularly useful.

The corresponding aglucones can be obtained by converting the glycoside isoflavones to aglucone isoflavones using conventional techniques. For example, acidic or enzymatic hydrolysis can be used to cleave the 1,4-glucoside bonds. Methods for converting glycoside isoflavones to aglucone isoflavones are disclosed in U.S. Pat. Nos. 5,919,921; 5,827,682 and Japanese Patent Application 258,669 to Obata, et al. In accordance with a particular conversion method, the purified isoflavone glycosides produced by the present invention can be converted to the aglycon form by prolonged refluxing in a mixture of methanol and hydrochloric acid (typically for 24 to 48 hours), followed by the addition of water and neutralization with sodium or potassium hydroxide.

In a further aspect of the present invention, the resulting aglycons can be further purified by dissolving them in solutions elevated to a pH of 10 to 14 (preferably 10.5 to 11.5) at moderate temperatures (typically below about 35° C.), filtering to remove insoluble impurities, and lowering the pH of the filtrate (preferably to a pH of 1 to 4) by addition of an excess of dilute mineral acid (e.g., hydrochloric or sulfuric acid) or an organic acid (e.g., formic or acetic acid). The precipitated isoflavone aglycons may be recovered by any acceptable technique including by filtration or centrifugation.

The present invention is illustrated in more detail by the following non-limiting examples. The examples are intended to be illustrative and should not be interpreted as limiting or otherwise restricting the scope of the invention in any way.

### EXAMPLE 1

50.0 grams of “Solgen 40” (containing 26.9% genistin, 11.9% daidzin, 2.0% glycetin; negligible aglycon content; genistin-to-daidzin ratio 2.3:1) was added to 75 ml of methanol and 15 ml of concentrated hydrochloric acid in an Erlenmeyer flask equipped with a magnetic stirring bar and reflux condenser. The mixture was heated on a hotplate to reflux (66° C.), maintained at reflux for 15 minutes, then cooled to 20° C. The resulting slurry was filtered at this temperature on a Buechner funnel through Whatman #541 filter paper. Filtration was very slow, as the viscosity of the slurry increased dramatically upon cooling. The off-white filter cake solids were washed with a modest volume of 5:1 methanol:HCl and dried in a vacuum oven at 80° C. to give 16.4 grams of off-white solids containing:

54.48% by wt. genistin (8.93 grams, or 66.3% of that present in the starting material)

9.47% by wt. daidzin (1.55 grams, or 26.1% of that present in the starting material)

63.95% total isoflavones; genistin-to-daidzin ratio=5.75:1

No glycetin or isoflavone aglycons were detected.

### EXAMPLE 2

50.0 grams of “Solgen 40” (containing 26.9% genistin, 11.9% daidzin, 2.0% glycetin; negligible aglycon content; genistin-to-daidzin ratio 2.3:1) was added to 250 ml of methanol and 50 ml of concentrated hydrochloric acid in an Erlenmeyer flask equipped with a magnetic stirring bar and reflux condenser. The mixture was stirred at room temperature for one hour, then filtered on a Buechner funnel through Whatman #541 filter paper. Filtration was very rapid, with no blinding. The off-white filter cake solids were washed with 20 ml of 5:1 methanol:HCl and dried in a vacuum oven at 80° C. to give 18.80 grams of off-white solids containing:

55.56% by wt. genistin (10.45 grams, or 77.6% of that present in the starting material)

10.47% by wt. daidzin (1.97 grams, or 33.1% of that present in the starting material)

Only a trace of glycetin was detected.

67.03% total isoflavones content; genistin-to-daidzin ratio=5.3:1

These solids were refluxed for 40 hours in 200 ml of methanol and 20 ml of concentrated HCl to convert the glycosides to aglycons. 100 ml of methanol was removed by distillation, then 100 ml of water was added. This slurry was cooled to 15° C. and filtered. The filter cake was slurried in 50 ml of water and the pH was adjusted to 10.8 by adding 4.7 grams of a 50% by wt. aqueous NaOH solution. This mixture was stirred for 30 minutes at room temperature, then filtered on a Buechner funnel to remove the insoluble dark brown particulates.

The filtrate (at room temperature) was charged to a separatory funnel and added dropwise to 100 ml of a vigorously stirred 5:1 mixture of water and concentrated hydrochloric acid heated to 65° C. in order to precipitate the purified aglycons. This slurry was cooled to 10° C. and filtered. The filter cake was dried in vacuo to give grams 7.68 of off-white solids containing:

83.5% genistein (6.41 grams contained, or 76% of that present in the feedstock, largely as glycoside)

16.5% daidzein (1.27 grams contained, or 35% of that present in the feedstock, largely as glycoside)

A trace of glycetein was detected.

Total isoflavone content >99.9%; genistein-to-daidzein ratio=5.06:1

### EXAMPLE 3

50.0 grams of ADM Novasoy (22.16% genistin+genistein; 18.56% daidzin+daidzein; 5.04% glycetin+glycetein; total aglycons 1.70%; total isoflavone content 45.39% by wt.; genistin-to daidzin ratio 1.2:1) was added to 250 ml of methanol and 50 ml of concentrated hydrochloric acid in an Erlenmeyer flask equipped with a magnetic stirring bar and reflux condenser. The mixture was heated on a hotplate to reflux (66° C.), maintained at reflux for 15 minutes, then cooled to 15° C. The resulting slurry was filtered at this temperature on a Buechner funnel through Whatman #541 filter paper. Filtration was very rapid, with no blinding. The off-white filter cake solids were washed with a modest volume of 5:1 methanol:HCl and dried in a vacuum oven at 80° C. to give 10.13 grams of off-white solids containing:

55.4% by wt. genistin (5.61 grams, or 50.7% of that present in the starting material)

15.65% by wt. daidzin (1.58 grams, or 17.1% of that present in the starting material)

0.86% by wt. glycetin (0.087 grams, or 3.4% of that present in the starting material)

72.00% total isoflavones; genistin-to-daidzin ratio=3.54:1

No isoflavone aglycons were detected.

### EXAMPLE 4

100 grams of ADM Novasoy (22.16% genistin+genistein; 18.56% daidzin+daidzein; 5.04% glycetin+glycetein; total aglycons 1.70%; total isoflavone content 45.39% by wt.; genistin-to daidzin ratio 1.2:1) was added to 500 ml of methanol and 100 ml of concentrated hydrochloric acid in an Erlenmeyer flask equipped with a magnetic stirring bar and reflux condenser. The mixture was stirred out for one hour at room temperature, then filtered at 20° C. on a Buechner funnel through Whatman #541 filter paper. Filtration was very rapid, with no blinding.

The off-white filter cake solids were washed with 20 ml of 5:1 methanol:HCl, charged to 200 ml methanol and 20 ml of concentrated HCl, and refluxed for 48 hours to convert the isoflavone glycosides to aglycons. The mixture was cooled to 20° C. and filtered. The filter cake was washed with 25 ml of acidified methanol and dried at 80° C. in vacuo to give 11.6 grams of off-white solids containing:

89.3% genistein (10.35 grams contained, or 75% of that present in the feedstock)

13.8% daidzein (1.60 grams contained, or 14% of that present in the feedstock)

0.3% glycetein (0.34 grams contained, or 11% of that present in the feedstock)

Genistein-to-daidzein ratio=6.5:1; total isoflavone content >99.9% by HPLC analysis

### EXAMPLE 5

10.0 gram aliquots of “Solgen 40” (containing 26.9% genistin, 11.9% daidzin, 2.0% glycetin; negligible aglycon content; genistin-to-daidzin ratio 2.3:1) were charged to a 100 ml Erlenmeyer flask equipped with a magnetic stirring bar. Varying amounts of solvents comprising glacial acetic acid (GAA) and various co-solvents were added and the resulting slurry was stirred at ambient temperature for 30 minutes, then filtered on a Buechner funnel through Whatman #541 paper. The filter cakes were washed with 10 milliliters of the solvent employed, dried in vacuo at 80° C., and subjected to HPLC analysis. The results are tabulated below:

| | SOLVENT- | | | | TOTAL |
|---|---|---|---|---|---|
| | TO- | | | | ISOFLAVONE |
| | SOLIDS | | | | GLYCOSIDE |
| | RATIO | GENISTIN | DAIDZIN | GLYCETIN | CONTENT OF |
| SOLVENT (vol/vol) % | (ml/g) | RECOVERY | RECOVERY | RECOVERY | FILTER CAKE |
| 100% GAA | 5:1 | 81.2% | 32.7% | 28.8% | 99%+ |
| 100% GAA | 2.5:1 | 87.4% | 40.5% | 52.9% | 88% |
| 80% GAA/20% water | 10:1 | 63.1% | 40.2% | 29.4% | 83% |
| 60% GAA/40% hexane | 5:1 | 90.5% | 34.8% | 15.0% | 93% |
| 70% GAA/30% hexane | 5:1 | 83.6% | 56.8% | 31.3% | 95% |
| 70% GAA/30% | 5:1 | 88.5% | 35.7% | 19.0% | 99%+ |
| cyclohexane | | | | | |
| 70% GAA/30% iso-octane | 5:1 | 85.9% | 33.0% | 15.9% | 99%+ |
| 80% GAA/20% octanoic | 5:1 | 82.8% | 33.0% | 18.0% | 86% |
| acid | | | | | |
| 60% GAA/40% ethyl | 5:1 | 88.5% | 56.1% | 23.5% | 88% |
| acetate | | | | | |
| 63.6% GAA/36.4% ethyl | 5:1 | 78.7% | 51.4% | 23.5% | 82% |
| acetate | | | | | |
| 70% GAA/30% ethyl | 5:1 | 77.0% | 52.3% | 47.1% | 78% |
| acetate | | | | | |
| 60% GAA/40% acetone | 5:1 | 98.2% | 54.7% | 80.0% | 69% |
| 80% GAA/20% acetone | 5:1 | 100.0% | 48.6% | 61.4% | 99%+ |
| 90% GAA/10% acetone | 5:1 | 91.2% | 43.2% | 57.1% | 95% |
| 70% GAA/20% | | | | | |
| acetone/10% hexane | 5:1 | 100% | 66.9% | 95.7% | 72% |

Having described the invention in detail and by reference to specific embodiments thereof, it will be apparent that numerous modifications and variations are possible without departing from the scope of the invention defined by the following claims.

What is claimed is:

1. A method for purifying glycosides of genistin and daidzin from impurities present in a soy isoflavones concentrate, comprising the steps of:
(a) digesting a soy isoflavones concentrate with an acidic solution; and
(b) separating insoluble solids from the acidic solution, wherein said solids are enriched in genistin and comprise glycosides of genistin and daidzin.

2. The method of claim 1 wherein the ratio of soy isoflavone concentrate to the acidic solution is between about 3:1 and 10:1 by weight.

3. The method of claim 1 wherein step (a) is conducted at a temperature of between about 10 and 100° C.

4. The method of claim 3 wherein step (a) is conducted at a temperature of between about 18 and 25° C.

5. The method of claim 1 wherein the acidic solution comprises glacial acetic acid or formic acid.

6. The method of claim 5 wherein the acidic solution comprises glacial acetic acid and an organic co-solvent selected from the group consisting of alcohols containing 1 to 12 carbons, aliphatic hydrocarbons containing 5 to 20 carbons, aromatic hydrocarbons containing 6 to 30 carbons, ketones containing 2 to 12 carbons, esters containing 3 to 30 carbons and mixtures thereof.

7. The method of claim 1 wherein the acidic solution comprises a mineral acid and an alcoholic co-solvent.

8. The method of claim 7 wherein said mineral acid is selected from the group consisting of hydrochloric acid, hydrobromic acid, sulfuric acid, phosphoric acid and mixtures thereof and said alcoholic co-solvent is selected from the group consisting of alcohols containing 1 to 8 carbons and mixtures thereof.

9. The method of claim 8 wherein said acidic solution comprises hydrochloric acid and methanol.

10. The method of claim 9 wherein said acidic solution comprises hydrochloric acid and methanol in a volume ratio of about 1:5.

11. A method of preparing aglycons of genistin and daidzin comprising the steps of:
(a) digesting a soy isoflavone concentrate with a first acidic solution;
(b) separating insoluble solids wherein said solids comprise glycosides of genistin and daidzin; and
(c) converting said glycosides to aglycons.

12. The method of claim 11 wherein step (c) comprises acidic or enzymatic hydrolysis.

13. The method of claim 12 wherein said acidic hydrolysis comprises refluxing said solids in a second acidic solution.

14. The method of claim 13 further comprising:
(d) separating insoluble solids from the second acidic solution, wherein the insoluble solids separated from the second acidic solution are enriched in genistin and comprise aglycons of genistin and daidzin.

15. The method of claim 11 further comprising the following steps after step (c):
(i) dissolving said aglycons in an alkaline aqueous solution to obtain an alkaline aqueous solution containing dissolved aglycons:
(ii) separating insoluble impurities from said alkaline aqueous solution;
(iii) acidifying said alkaline aqueous solution to precipitate the aglycons as insoluble solids; and
(iv) separating said insoluble solids from said acidified solution, wherein the insoluble solids removed from said acidified solution are enriched in genistin and comprise aglycons of genistin and daidzin.

16. The method of claim 15 wherein said insoluble solids consist essentially of aglycons of genistin and daidzin.

17. The method of claim 15 wherein the pH of the alkaline aqueous solution is between about 10 and 14 and wherein the alkaline aqueous solution includes at least one primary cation selected from the group consisting of sodium, potassium, calcium, and ammonium.

18. The method of claim 15 wherein said acidifying step comprises adjusting the pH of the solution to a pH value of between about 1 and about 7 with a mineral acid selected from the group consisting of hydrochloric acid, hydrobromic acid, sulfuric acid, phosphoric acid, and mixtures thereof.

19. The method of claim 15 wherein the alkaline aqueous solution in step (ii) has a pH of from about 10.5 to about 11.5, the acidified solution of step (iii) has a pH of from about 1 to about 4, and where steps (i) through (iv) are performed at temperatures below about 35° C.

20. The method of claim 11 wherein the ratio of soy isoflavone concentrate to the first acidic solution is between about 3:1 and 10:1 by weight.

21. The method of claim 11 wherein step (a) is conducted at a temperature of between about 10 and 100° C.

22. The method of claim 11 wherein the first acidic solution comprises glacial acetic acid or formic acid.

23. The method of claim 15 wherein at least one of the first and second acidic solutions comprises a mineral acid and an alcoholic co-solvent.

24. The method of claim 23 wherein said mineral acid is selected from the group consisting of hydrochloric acid, hydrobromic acid, sulfuric acid, phosphoric acid and mixtures thereof and said alcoholic co-solvent is selected from the group consisting of alcohols containing 1 to 8 carbons and mixtures thereof.

25. The method of claim 24 wherein said mineral acid comprises hydrochloric acid and said co-solvent comprises methanol.

26. The method of claim 25 wherein said hydrochloric acid and methanol are present in a volume ratio of about 1:5.

27. A method of purifying glycosides of genistin, daidzin, and glycetin from impurities present in a soy isoflavone concentrate comprising the steps of:
(a) digesting a soy isoflavone concentrate with an acidic solution comprising glacial acetic acid in conjunction with an organic solvent that reduces the polarity of glacial acetic acid, thereby increasing the solubilities of daidzin and glycetin; and
(b) separating the insoluble solids from said acidic solution, wherein said solids comprise glycosides of genistin, daidzin, and glycetin.

28. The method of claim 27 wherein said solids consist essentially of genistin, daidzin, and glycetin.

29. The method of claim 27 wherein said organic solvent is selected from the group consisting of alcohols containing 1 to 12 carbons, aliphatic hydrocarbons containing 1 to 20 carbons, aromatic hydrocarbons containing 6 to 30 hydrocarbons, ketones containing 2 to 12 carbons, esters containing three to 30 carbons, and mixtures thereof.
